(19) United States
(12) Patent Application Publication
(10) Pub. No.: US 2024/0299267 A1
(43) Pub. Date: Sep. 12, 2024

(54) FRAGRANCED COSMETIC COMPOSITION COMPRISING AT LEAST ONE FERULIC ACID DERIVATIVE, A DYESTUFF AND A FRAGRANCING MATERIAL, AND PROCESS FOR TREATING KERATIN MATERIALS AND/OR CLOTHING USING THE COMPOSITION

(71) Applicant: L'OREAL, Paris (FR)

(72) Inventor: Christophe BOBIN, Chevilly Larue (FR)

(21) Appl. No.: 18/260,143

(22) PCT Filed: Dec. 28, 2021
(86) PCT No.: PCT/EP2021/087747

§ 371 Date: Jun. 30, 2023

(30) Foreign Application Priority Data
Dec. 30, 2020 (FR) 2014277

(51) Int. Cl. A61K 8/365; A61Q 13/00

(52) U.S. Cl. CPC A61K 8/365; A61Q 13/00; A61K 2800/43

(57) ABSTRACT

The present invention relates to a fragranced composition comprising: a) at least one compound of formula (I), or a geometrical isomer thereof, an organic or mineral acid or base salt thereof, or a solvate thereof, in particular hydrates:

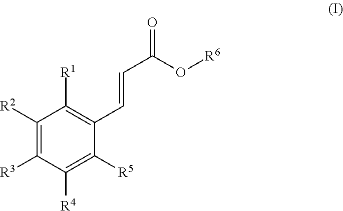

b) at least one dyestuff; and c) at least one fragrancing substance.

### TECHNICAL FIELD

The present invention is directed towards proposing a novel fragranced cosmetic composition, for the field of fragrancing keratin materials and/or clothing, but also for the cosmetic care or treatment of keratin materials.

In general, the formulation of environmentally friendly cosmetic products has become a major challenge for meeting new consumer expectations, in particular regarding natural and/or eco-friendly products, i.e. products whose design and development take into account their environmental impacts.

It is thus common practice to seek to replace synthetic compounds present in cosmetic compositions with natural ingredients and/or ingredients of natural origin.

These considerations have notably given rise to the development of green chemistry, also known as sustainable chemistry or ecological chemistry, which envisages the implementation of principles for reducing and eliminating the use or generation of environmentally unfriendly substances.

The guiding principle of “Green Chemistry”, put forward in 1991, is to reduce or eliminate at source the use of hazardous substances in the design of new products.

It was introduced in 1998 by the American chemists Paul Anastas and John C. Warner, who were members of the EPA (“United States Environmental Protection Agency”), who proposed twelve principles of green chemistry in the publication “Green chemistry: theory and practice”, Oxford Science, New York, P. T. Anastas et al., (1998). The indicators of green chemistry, such as the E-factor or the economy of atoms, make it possible to measure the various aspects of a chemical process by referring to the principles of green chemistry. The publications Kirk-Othmer, Encyclopedia of Chemical Technology, Green Chemistry, Michael A. Matthews, 2013, volume 12, pages 799 to 818, and Ibid., Green Chemistry, Applications, Albert S. Matlack, pages 1 to 33, illustrate such indicators.

These environmental problems concern all cosmetic products, notably fragranced compositions.

A perfume is the result of a combination of different odorous substances which each provide a specific diffusing odour or “note” and which respectively evaporate at different periods. More precisely, each perfume has what is known as a “head note”, which is the odour that diffuses first when the perfume is applied or when the receptacle containing it is opened, a “heart or body note”, which corresponds to the full fragrance (given off for a few hours after the “head note”) and a “base note”, which is the most persistent odour (given off for several hours after the “body note”). The persistence of the base note corresponds to the remanence of the perfume.

Human beings have always sought to perfume themselves and to perfume the objects surrounding them or their environments, both to mask strong and/or unpleasant odours and to give a nice odour.

It is common practice to incorporate perfume into a certain number of products or compositions, in particular cosmetic and dermatological compositions such as eaux fraîches, eaux de toilette, eaux de parfum, perfume elixirs or extracts, aftershave lotions, care fluids or two-phase lotions.

For aesthetic and manufacturing cost reasons, perfumes are coloured by adding an effective amount of dyestuff to the formulation support (which is generally alcoholic or aqueous-alcoholic) rather than tinting or lacquering the bottle, which is a more expensive industrial operation.

For obvious reasons, the colour developed in these fragranced formulations must remain stable both over time and when exposed to light.

### PRIOR ART

In order to ensure the stability of fragranced formulations, it has already been proposed to incorporate therein a filtering system and/or an antioxidant system.

Thus, a certain number of documents in the cosmetics field, notably EP 1 897 592 and WO 2005/042 828, propose fragranced compositions, notably including dyes, comprising combinations of UV-screening agents as stabilizer, in particular of the alkyl β,β′-diphenylacrylate or alkyl α-cyano-β,β′-diphenylacrylate type, such as octocrylene, dibenzoylmethane derivatives, cinnamates or alternatively camphor and triazine derivatives. However, certain screening agents such as alkyl β,β′-diphenylacrylate or alkyl α-cyano-β,β′-diphenylacrylate compounds, for instance octocrylene, are not always satisfactory for stabilizing the tints while at the same time maintaining the odour of the perfumes, notably in the yellow and violet ranges.

The reason for this is that certain screening agents may leave an unpleasant odour in the formulation. Synthetic screening agents, such as ethylhexyl methoxycinnamate, are also unsatisfactory for stabilizing the dyestuffs, in particular the dyes and/or pigments, of a fragrancing solution. Notably, they prove to be less efficient in terms of conserving the colour intensity and/or the chromaticity of the coloured solution with respect to light and/or over time.

Moreover, the piperidinol derivatives used in some of these compositions have the drawback of generating yellowing in eaux de toilette and also a spurious odour with a “chemical” connotation.

Also, certain particular benzotriazole and/or triazine compounds have already been described for stabilizing cosmetic compositions, such as eaux de toilette. Combinations of these benzotriazole compounds with other particular sunscreens have also been defined for perfume-stabilizing purposes. However, these benzotriazole compounds, in particular bumetrizole, are difficult to dissolve in eaux de toilette and have a tendency to recrystallize over time, in addition to having low stabilizing power.

The combination of a UV-screening agent such as aminobenzophenone with other particular screening agents has moreover been proposed for stabilizing fragranced compositions.

However, aminobenzophenone screening agents give a yellow colour that is not desirable for pale-coloured fragranced formulations other than yellow.

Moreover, certain dyestuffs contained in coloured fragranced compositions may leave traces when they are applied to keratin materials and/or clothing, and in particular may have the drawback of staining clothing.

Besides the potential problems mentioned above associated with their presence, generally speaking, the sunscreens proposed to date, such as ethylhexyl methoxycinnamate, are synthetic.

However, as mentioned previously, an ever-increasing number of users are in search of coloured fragranced compositions that are “less chemical” and more environmentally friendly. These users may perceive synthetic compounds negatively, notably as compounds that are harmful to their health and to the environment. Thus, coloured fragranced compositions comprising synthetic sunscreens are not entirely satisfactory to consumers who favour natural compounds or compounds of natural origin over synthetic compounds.

What is more, these coloured fragranced compositions generally do not respect the principles of green chemistry.

There is thus every interest in finding a means for stabilizing the colour of fragrancing compositions, if possible with natural products or products of natural origin.

### DISCLOSURE OF THE INVENTION

The need thus remains to find novel coloured fragranced formulations, notably aqueous or aqueous-alcoholic formulations, which remain stable over time and under the effects of light. In particular, the need remains for coloured fragranced cosmetic compositions for which the evolution of the organoleptic properties, namely the odour and/or the colour, in particular at least the colour, is controlled over time, and this being achieved if possible by means of compounds which adhere to at least one of the twelve principles of green chemistry.

In addition, there is a need for such compositions whose formulation adheres to at least one of the twelve principles of green chemistry, in particular comprising ingredients derived from green chemistry.

Finally, the need remains to propose coloured fragranced cosmetic compositions which leave few or no traces, in particular on clothing, when they are used.

The present invention is specifically directed towards meeting these needs.

### SUMMARY OF THE INVENTION

Thus, according to a first of its aspects, the present invention relates to a fragranced composition, notably a cosmetic composition, comprising:
a) at least one compound of formula (I), or a geometrical isomer thereof, an organic or mineral acid or base salt thereof, or a solvate thereof, in particular hydrates:

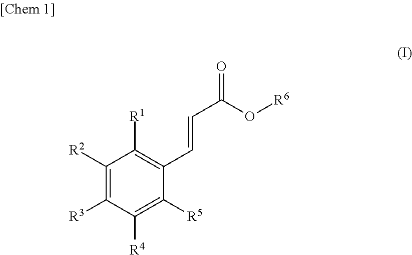

in which formula (I):
   R¹, R⁴ and R⁵ represent, independently of each other, a hydrogen atom or a hydroxyl group, preferably a hydrogen atom;
R² represents a hydrogen atom, a hydroxyl group or a (C₁-C₆)alkoxy group, in particular a (C₁-C₄)alkoxy group, and preferably a methoxy;
R³ represents a hydroxyl group or a (C₁-C₆)alkoxy group, preferably a hydroxyl group; and
R⁶ represents an atom or a group chosen from: i) hydrogen, ii) (C₁-C₆)alkyl, in particular a (C₁-C₄)alkyl group, preferably methyl or ethyl, optionally substituted with one or more groups chosen from hydroxyl, amino, carboxyl and/or amido, preferably hydroxyl and/or carboxyl, iii) cycloalkyl, optionally substituted with one or more groups chosen from (C₁-C₁₀)alkyl, (C₂-C₁₀)alkenyl, hydroxyl, amino, carboxyl and/or amido, preferably hydroxyl and/or carboxyl, and iv) glycosyl or glycosyloxy,
b) at least one dyestuff; and
c) at least one fragrancing substance.

The inventors have found, surprisingly, that the combination of a compound of formula (I) as defined above, in a fragranced composition, with at least one fragrancing substance and at least one dyestuff allows efficient stabilization of said composition.

Specifically, as emerges from the examples given below, the composition according to the invention is stable, in the sense that the evolution of the colour of the composition remains low over time, even under conditions simulating accelerated ageing of the composition, notably in aqueous-alcoholic medium, and with respect to light.

Thus, according to another of its aspects, the invention also relates to the use of a compound of formula (I), as a stabilizer in a fragranced composition, notably a cosmetic composition, notably an aqueous or aqueous-alcoholic composition, also comprising at least one dyestuff, and at least one fragrancing substance.

A composition according to the invention is in particular intended for the cosmetic treatment of keratin materials.

Thus, according to another of its aspects, the invention relates to a cosmetic process for treating keratin materials, in particular the skin, comprising at least one step of applying a composition according to the invention to said keratin materials.

The invention also relates to a cosmetic process for treating keratin materials, in particular the skin, or clothing, comprising at least one step of applying a fragranced composition according to the invention to said keratin materials and/or said clothing.

It is understood that the cosmetic treatment processes targeted in the present patent application are non-therapeutic.

A composition according to the invention is notably intended to be used in order to fragrance keratin materials and/or clothing.

Thus, according to another of its aspects, the invention also relates to a process for fragrancing keratin materials, notably the skin, and/or clothing, comprising the application of the composition as defined above to said keratin materials and/or to said clothing.

Advantageously, the composition may be applied by spraying, notably using a spray.

Specifically, the inventors have found that spraying of the fragranced composition according to the invention reduces the risks of forming stains on clothing during its application, when compared with other application methods.

In the context of the present invention, and unless otherwise indicated, the following definitions apply:

For the purposes of the present invention, the term “fragranced composition” or “fragrancing composition” is intended to denote any composition which leaves a perfume on keratin materials after application.

For the purposes of the present invention, the term “keratin materials” is notably intended to denote the skin, the lips, the hair, the scalp, the eyelashes and the eyebrows or else the nails, in particular the skin and/or the lips, and preferably the skin.

In other words, a fragranced composition according to the invention is said to be “coloured”. The term “coloured composition” means a composition which appears to the human eye to have a colour of the visible spectrum, i.e. which appears to the eye as different from white or colourless, namely a composition which absorbs in the yellow, orange, red, purple, violet, blue and green ranges. Such a composition may be opaque or clear, preferably clear.

For the purposes of the present invention, the term “clear” means that the composition is transparent, i.e. it allows light to pass through and objects that are located behind it are seen in sharp definition.

In particular, to evaluate the transparency of a composition according to the invention, a conventional commercial untinted, transparent borosilicate, soda-lime or neutral glass test tube such as a test tube made of Pyrex® or Duran® glass, 16 cm tall and 1.6 cm in diameter is taken and is filled to 14 cm of said tube (the bottom of the meniscus of the composition in the tube is located 14 cm from the bottom of the tube), and a page of published international patent application (in A4 format) is placed behind it at a distance of 1 cm; and by placing the eyes at a distance (reading focal length relative to the test tube) of between 20 cm and 40 cm, such as 30 cm, and perpendicular to the middle of the composition of said tube (about 7 cm from the bottom of said tube), an evaluation is made as to whether or not it is possible to read distinctly said page of the published international patent application, notably the first page, through the composition in said tube. It is understood that the tester has good vision, or that he is equipped with spectacles, contact lenses or any other optical devices that he normally wears for reading. Use may be made, for example, of patent application WO 2018/104 428 and evaluate the sharpness, i.e. the ability to read the 31 lines of page 13 to perform the test or, better still, to perform the reading test on the first page.

The term “dyestuff” is intended to denote any compound that is capable of colouring the fragranced composition, i.e. any compound which absorbs in the visible spectrum, in particular so as to appear to the human eye to have a colour such as yellow, orange, red, violet, blue or green.

As visual colour and absorption wavelength associated with said colour, mention may be made of the following colours: “yellow”=λₘₐₓ greater than 400 nm up to 440 nm limits inclusive, “orange”=λₘₐₓ greater than 440 nm up to 490 nm limits inclusive, “red”=λₘₐₓ greater than 490 nm up to 520 nm limits inclusive, “purple”=λₘₐₓ greater than 520 nm up to 560 nm limits inclusive, “violet”=λₘₐₓ greater than 560 nm up to 580 nm limits inclusive, “blue”=λₘₐₓ greater than 580 nm up to 620 nm limits inclusive, “blue-green”=kₘₐₓ greater than 620 nm up to 650 nm limits inclusive, and “green”=λₘₐₓ greater than 650 nm up to 780 nm limits inclusive.

A fragranced coloured composition may be in any form that is suitable for its application to keratin materials and/or clothing. It may notably be in liquid or solid form, for example in powder form.

The term “powder” means a composition in pulverulent form, which is preferably essentially free of dust. In other words, the particle size distribution of the particles is such that the weight content of particles which have a size of less than or equal to 50 μm (content of fines), preferably less than or equal to 10 μm (content of fines) is advantageously less than or equal to 5%, preferably less than 2% and more particularly less than 1% (particle size evaluated using a Retsch AS 200 Digit particle size analyser; oscillation height: 1.25 mm/screening time: 5 minutes). Advantageously, the particle size is between 10 μm and 500 μm. The powder of solid natural material(s) may be screened to obtain particles with upper limit sizes corresponding to the orifices or mesh sizes of the screen particularly between 35 and 80 mesh (US). According to a particular mode of the invention, the size of the particles of the powder of solid natural material(s) is fine. According to the invention, this more particularly means a particle size of less than or equal to 900 μm. Preferentially, the powder is constituted of fine particles between 7 and 700 μm and better still between 100 nm and 500 μm in size. For the purposes of the present invention, the term “fragrancing substance” means any odorous starting material or aroma that is capable of giving off a pleasant odour, in particular as defined in the continuation of the text. For the purposes of the present invention, “fragrancing”, “odorous” or “odoriferous” substances are synonymous.

A composition according to the invention is generally suitable for application to keratin materials, in particular topical application to the skin, and thus generally comprises a physiologically acceptable medium, i.e. a medium that is compatible with the skin.

It is preferably a cosmetically acceptable medium, i.e. a medium which has a pleasant colour, odour and feel and which does not cause any unacceptable discomfort, i.e. stinging, tautness or redness, liable to discourage the user from applying this composition.

For the purposes of the present invention, a “stabilizer” is intended to denote a compound that is capable of stabilizing the fragranced composition comprising same, in particular in terms of conservation of its organoleptic properties with respect to external attacking factors, notably the light or temperature differences, in particular the colour and/or the odour of said composition.

A (Cₓ-C_{z})alkyl group represents a linear or branched hydrocarbon-based chain comprising from x to z carbon atoms. For example, a (C₁-C₆)alkyl group represents a linear or branched hydrocarbon-based chain comprising from 1 to 6 carbon atoms.

A (Cₓ-C_{z})alkenyl group represents a linear or branched hydrocarbon-based chain comprising from x to z carbon atoms, and comprising one or more conjugated or non-conjugated unsaturations, preferably only one unsaturation. For example, a (C₂-C₁₀)alkenyl group represents a linear or branched hydrocarbon-based chain comprising from 2 to 10 carbon atoms and comprising one or more unsaturations.

A (Cₓ-C_{z})alkoxy group represents a radical —O—(Cₓ-C_{z})alkyl in which the (Cₓ-C_{z})alkyl group is as defined previously.

The term “cycloalkyl” means a fused or non-fused, more preferentially fused, saturated or unsaturated, preferably saturated, non-aromatic, monocyclic or polycyclic carbocycle (cyclic hydrocarbon-based group), preferably of between 2 and 5 rings, comprising from 5 to 42 carbon atoms, in particular comprising from 6 to 10 carbon atoms.

The term “aryl” means a monocyclic or fused or non-fused polycyclic carbocycle comprising from 6 to 22 carbon atoms, and in which at least one ring is aromatic; preferentially, the aryl radical is a phenyl, biphenyl, naphthyl, indenyl, anthracenyl or tetrahydronaphthyl, preferably phenyl.

The term “glycosyl” group is intended to denote a monovalent radical obtained from a monosaccharide or polysaccharide by replacement of one of the hemiacetal hydroxyl groups of said monosaccharide or polysaccharide with a covalent bond with the compound bearing the group.

The term “glycosyloxy” group means an —O-glycosyl radical, in which the glycosyl group is as defined previously.

Preferentially, the compound of formula (I) is chosen from natural compounds or compounds of natural origin.

For the purposes of the present invention, the term “natural” means that the dyestuff is obtained directly from the earth or the soil, or from plants or animals, via, where appropriate, one or more physical processes, for instance milling, refining, distillation, purification or filtration, or else from a biotechnological process, notably obtained from microbiological or cell cultures, for example from fungi or from bacteria. Among the “natural” dyes or pigments are compounds that are present in nature and that can be reproduced by chemical (semi)synthesis.

For the purposes of the present invention, the term “of natural origin” means that the dyestuff is obtained from a natural substance which has undergone one or more associated chemical or industrial treatments, giving rise to changes which do not affect the essential qualities of this substance. As non-limiting examples of additional chemical and industrial treatments bringing about modifications which do not affect the essential qualities of a natural compound, mention may be made of those permitted by the controlling bodies, such as Ecocert (Reference system for biological and ecological cosmetic products, January 2003), or defined in recognized handbooks in the field, such as Cosmetics and Toiletries Magazine, 2005, volume 120, 9:10.

According to the invention, a compound is considered to be natural or of natural origin when it is predominantly composed of natural constituents, that is to say when the weight ratio of natural constituents to non-natural constituents which make up the compound is greater than 1.

More advantageously, the compound of formula (I) is a “green” compound, that is to say that it adheres to at least one of the twelve principles of green chemistry.

For the purposes of the present invention, the term “geometrical isomer” is understood to denote any molecule whose constitution is identical to that of formula (I) defined above, but in which the spatial organization of the atoms is different. In particular, they may be conformational stereoisomers, i.e. compounds which differ in their rotation around a single bond, or configurational stereoisomers, in particular enantiomers or diastereoisomers.

The term “anionic counterion” means an anion or an anionic group derived from an organic or mineral acid salt which counterbalances the cationic charge of the dye; more particularly, the anionic counterion is chosen from: i) halides such as chloride or bromide; ii) nitrates; iii) sulfonates, including C₁-C₆ alkylsulfonates: Alk-S(O)₂O— such as methylsulfonate or mesylate and ethylsulfonate; iv) arylsulfonates: Ar—S(O)₂O⁻ such as benzenesulfonate and toluenesulfonate or tosylate; v) citrate; vi) succinate; vii) tartrate; viii) lactate; ix) alkyl sulfates: Alk-O—S(O)O— such as methyl sulfate and ethyl sulfate; x) aryl sulfates: Alk-O—S(O)O— such as benzene sulfate and toluene sulfate; xi) alkoxy sulfates: Alk-O—S(O)₂O— such as methoxy sulfate and ethoxy sulfate; xii) aryloxy sulfates: Ar—O—S(O)₂O—, xiii) phosphates O═P(OH)₂—O⁻, O═P(O⁻)₂—OH O═P(O—)₃, HO—[P(O)(O⁻)]_{w}—P(O)(O⁺)₂ with w being an integer; xiv) acetate; xv) triflate; and xvi) borates such as tetrafluoroborate, xvii) disulfate (O═)₂S(O⁻)₂ or SO₄²⁻ and monosulfate HSO₄⁻; the anionic counterion, derived from the organic or mineral acid salt, ensures the electrical neutrality of the molecule.

Moreover, the addition salts that may be used in the context of the invention are notably chosen from addition salts with a cosmetically acceptable base such as the basifying agents as defined below, for instance alkali metal hydroxides such as sodium hydroxide, potassium hydroxide, aqueous ammonia, amines or alkanolamines.

The expression “at least one” is equivalent to “one or more”.

The term “inclusive” for a range of concentrations means that the limits of the range are included in the defined interval.

For the purposes of the present invention, the term “organic or mineral acid or base salt” is intended to denote a salt obtained between an ionic form of a compound of formula (I) and a corresponding counterion. In particular, such a salt is obtained by addition of an organic or mineral acid or base, which is notably cosmetically acceptable, to the compound of formula (I). Examples of mineral bases that may be mentioned include hydroxides or carbonates of alkali metals or alkaline-earth metals, such as sodium, potassium, calcium, magnesium or lithium, or ammonium. Examples of organic bases that may be mentioned include amines or alkanolamines. Examples of acids that may be mentioned include hydrochloric acid, hydrobromic acid, sulfuric acid, alkylsulfonic acids, arylsulfonic acids, citric acid, succinic acid, tartaric acid, lactic acid, alkoxysulfinic acids, aryloxysulfinic acids, phosphoric acid and acetic acid.

For the purposes of the present invention, the term “solvate” is intended to denote the form of the compound of formula (I) when it is associated with a solvent. The solvates include conventional solvates formed during the process for the preparation of the compound of formula (I). Examples of solvates are those obtained in the presence of water or of a linear or branched alcohol, in particular ethanol or isopropanol.

The terms “between . . . and . . . ”, “comprises from . . . to . . . ”, “formed from . . . to . . . ” and “ranging from . . . to . . . ” should be understood as being inclusive of the limits, unless otherwise specified. Other characteristics, variants and advantages of the compositions according to the invention will emerge more clearly on reading the description and the examples that follow.

### DETAILED DESCRIPTION

### Compounds of Formula (I)

As mentioned previously, a fragranced composition according to the invention comprises at least one compound of formula (I), or a geometrical isomer thereof, an organic or mineral acid or base salt thereof, or a solvate thereof, in particular hydrates:

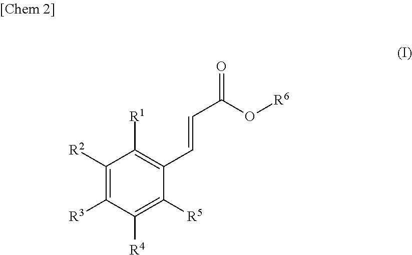

in which formula (I):
R¹, R⁴ and R⁵ represent, independently of each other, a hydrogen atom or a hydroxyl group, preferably a hydrogen atom;
R² represents a hydrogen atom, a hydroxyl group or a (C₁-C₆)alkoxy group, preferably a (C₁-C₄)alkoxy group, in particular methoxy;
R³ represents a hydroxyl group or a (C₁-C₆)alkoxy group, preferably a hydroxyl group; and
R⁶ represents an atom or a group chosen from: i) hydrogen, ii) (C₁-C₆)alkyl, preferably (C₁-C₄)alkyl, in particular methyl or ethyl, optionally substituted with one or more groups chosen from hydroxyl, amino, carboxyl and/or amido, preferably hydroxyl and/or carboxyl, iii) cycloalkyl, optionally substituted with one or more groups chosen from (C₁-C₁₀)alkyl, (C₂-C₁₀)alkenyl, hydroxyl, amino, carboxyl and/or amido, preferably hydroxyl and/or carboxyl, and iv) glycosyl or glycosyloxy.

According to a particular embodiment of the invention, R⁶ represents an atom or a group chosen from i) hydrogen and ii) (C₁-C₆)alkyl, as defined above.

According to another particular embodiment of the invention, R⁶ represents iii) a cycloalkyl group, and preferentially the cycloalkyl group is a saturated or unsaturated, preferably saturated, non-aromatic monocyclic group comprising from 5 to 7 carbon atoms and is optionally substituted with one or more (C₁-C₄)alkyl groups and/or with one or more groups chosen from hydroxyl, amino, carboxyl and/or amido, preferably hydroxyl and/or carboxyl. In particular, the cycloalkyl group is cyclohexyl, optionally substituted with 1 to 5 hydroxyl and/or carboxyl groups. In particular, the cycloalkyl group is cyclohexyl and is optionally substituted with one or more groups chosen from hydroxyl, amino, carboxyl and/or amido, preferably hydroxyl and/or carboxyl. More particularly, the cycloalkyl group is cyclohexyl, optionally substituted with 1 to 5 hydroxyl and/or carboxyl groups.

According to another particular embodiment of the invention, R⁶ represents iii) a cycloalkyl group, said group being polycyclic, preferably comprising between 2 and 5 fused rings, comprising from 12 to 32 carbon atoms and being optionally substituted; in particular, the cycloalkyl is a polycyclic group comprising between 4 and 5 fused rings, said rings being 3-, 5- or 6-membered and containing 16 to 20 carbon atoms, better still 17 or 18 carbon atoms, said cycloalkyl being optionally substituted with one or more linear or branched (C₁-C₁₀)alkyl or linear or branched (C₂-C₁₀)alkenyl groups.

Preferably, the compound is of formula (I) in which:
R¹, R⁴ and R⁵ represent hydrogen atoms;
R² represents a hydroxyl group or a (C₁-C₆)alkoxy group, preferably a (C₁-C₄)alkoxy group, in particular methoxy;
R³ represents a hydroxyl group or a (C₁-C₆)alkoxy group; and
R⁶ represents an atom or group chosen from i) hydrogen or ii) (C₁-C₆)alkyl, preferably (C₁-C₄)alkyl,
and also the geometrical isomers thereof, the organic or mineral acid or base salts thereof,
and the solvates thereof such as hydrates.

More preferentially, the compound is of formula (I) in which:
R¹, R⁴ and R⁵ represent hydrogen atoms;
R² represents a (C₁-C₄)alkoxy group, preferably methoxy;
R³ represents a hydroxyl group; and
R⁶ represents an atom or group chosen from i) hydrogen, and ii) methyl or ethyl, and preferably i) a hydrogen atom;
and also the geometrical isomers thereof, the organic or mineral acid or base salts thereof,
and the solvates thereof such as hydrates.

According to a particularly preferred embodiment, the compound of formula (I) is of formula (I′) below:

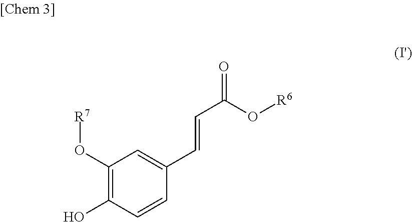

in which formula (I′):
R⁶ is as defined above; preferably, R⁶ represents a hydrogen atom or a polycyclic cycloalkyl group comprising between 3 and 5 fused saturated rings, comprising between 16 and 20 carbon atoms, substituted with at least one and preferably with at least two (C₁-C₄)alkyl groups, preferably methyl, and with at least one, preferably one, branched (C₅-C₁₀)alkenyl or (C₅-C₁₀)alkyl group; and
R⁷ represents a (C₁-C₆)alkyl and preferably (C₁-C₄)alkyl group, preferably a methyl group.

The compounds of formulae (I) and (I′) in accordance with the invention are preferably chosen from natural compounds or compounds of natural origin.

They are preferably “green” compounds.

Preferably, the compound of formula (I) is chosen from ferulic acid and derivatives thereof. The term “ferulic acid derivative” is more precisely intended to denote the esters thereof and/or the cosmetically acceptable salts thereof, preferably the esters thereof.

As particular examples of ferulic acid esters, mention may be made of methyl ferulate, ethyl ferulate, chlorogenic acid, isopropyl ferulate or ferulic acid esters of sterols, such as oryzanol, notably γ-oryzanol.

Ferulic acid is more particularly preferred.

This compound, also known as 3-(4-hydroxy-3-methoxyphenyl)prop-2-enoic acid, or 3-(4-hydroxy-3-methoxyphenyl)acrylic acid, or 3-methoxy-4-hydroxycinnamic acid, is a hydroxycinnamic acid or coumaric acid derivative.

It is found in numerous plant extracts, in particular in tree bark, poplar buds, asparagus, fennel, artichoke, olives, coffee grains, peanuts, apples and oranges, and also in plants such as Lilianae, such as rice, wheat, barley or oat.

According to another embodiment, oryzanol, and in particular γ-oryzanol, is particularly preferred.

The compounds of formulae (I) and (I′) may be obtained via any method known to those skilled in the art.

For example, ferulic acid may be obtained by biosynthesis by methoxylation of caffeic acid using the enzyme caffeic acid O-methyltransferase. It may also be obtained by extraction from a plant extract, in particular wheat bran, for example using concentrated bases such as sodium hydroxide and potassium hydroxide.

γ-Oryzanol may be obtained by extraction using an organic solvent, notably from rice bran. The compounds of formula (I) or (I′) defined above may optionally be introduced into a fragranced composition according to the invention in the form of a plant extract. For example, ferulic acid may be used in the form of an extract of rice, wheat, barley or oat bran, in particular of rice bran, and γ-oryzanol may be used in the form of an extract of rice bran. Mention may notably be made of oryzanol, preferentially γ-oryzanol.

A certain number of compounds of formula (I) or (I′) are also commercially available, for instance the ferulic acid available under the commercial reference Oryza Ferulix® from the company Oryza Oil & Fat Chemicals, or γ-oryzanol available under the commercial reference Oryzanol® from the company Tsuno Rice Fine Chemicals, γ-Oryzanol® from the company Ikeda or Oryzanolgamma V® from the company Ichimaru Pharcos.

Preferably, a composition according to the invention comprises a compound of formula (I) chosen from ferulic acid and derivatives thereof, and in particular chosen from ferulic acid, γ-oryzanol and mixtures thereof.

A fragranced composition according to the invention may comprise from 0.01% to 5.0% by weight, in particular from 0.03% to 2.0% by weight, preferably from 0.05% to 1.0% by weight and more preferentially from 0.1% to 0.8% by weight of compound(s) of formula (I), relative to the total weight of said composition.

As mentioned above, the compound of formula (I) may be used in the form of a plant extract also known as a “crude raw material”, comprising one or more compounds of formula (I) as defined previously. Thus, a fragranced composition according to the invention may comprise from 2.0% to 20% by weight of plant extract containing at least one compound of formula (I), relative to the total weight of said composition.

### Dyestuff

As mentioned previously, a fragranced composition according to the invention comprises at least one dyestuff.

As mentioned previously, a dyestuff is capable of colouring the fragranced composition by absorbing in the visible spectrum.

As visual colour and absorption wavelength associated with said colour, mention may be made of the following colours: “yellow”=λₘₐₓ greater than 400 nm up to 440 nm limits inclusive, “orange”=λₘₐₓ greater than 440 nm up to 490 nm limits inclusive, “red”=λₘₐₓ greater than 490 nm up to 520 nm limits inclusive, “purple”=λₘₐₓ greater than 520 nm up to 560 nm limits inclusive, “violet”=λₘₐₓ greater than 560 nm up to 580 nm limits inclusive, “blue”=λₘₐₓ greater than 580 nm up to 620 nm limits inclusive, “blue-green”=λₘₐₓ greater than 620 nm up to 650 nm limits inclusive, and “green”=λₘₐₓ greater than 650 nm up to 780 nm limits inclusive.

The dyestuff may be chosen from water-soluble dyes, liposoluble dyes and colouring particulate materials, in particular pigments, nacres and materials with an optical effect, preferably from water-soluble dyes, liposoluble dyes and natural pigments or pigments of natural origin.

Preferably, the dyestuff is different from the compound of formula (I) as defined above.

### Water-Soluble Dye

For the purposes of the invention, the term “water-soluble dye” denotes any natural or synthetic, generally organic dye, which is soluble in an aqueous phase or in water-miscible solvents. In particular, a dye is water-soluble when its solubility in water, measured at 25° C., is at least equal to 0.01 g/L.

As water-soluble dyes that are suitable for use in the invention, mention may be made notably of synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanine (beetroot), carmine, copper chlorophyllin, methylene blue, ortho-diphenol derivatives, in particular anthocyanins (red radish, red cabbage, violet sweet potato, violet corn, black carrot, hibiscus, blue pea and elder), caramel, sandalwood, gardenia, spirulina and riboflavin.

### Liposoluble Dye

For the purposes of the invention, the term “liposoluble dye” denotes any natural or synthetic, generally organic dye, which is soluble in an oily phase or in solvents that are miscible with a fatty substance. As liposoluble dyes that are suitable for use in the invention, mention may notably be made of synthetic or natural liposoluble dyes, for instance DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto and curcumin.

### Colouring Particulate Materials

The colouring particulate materials may be pigments, nacres and/or particles with metallic tints.

### Pigments

For the purposes of the present invention, the term “pigments” is intended to denote white or coloured, mineral or organic solid particles.

Pigments are naturally insoluble in the liquid hydrophilic and lipophilic phases usually employed in cosmetics. Alternatively, they may be rendered insoluble by formulation in lake form, where appropriate.

More particularly, the pigment has little or no solubility in aqueous-alcoholic media. Examples of pigments that may be mentioned include natural organic and inorganic pigments such as those defined and described in the article by Hunger et al., Ullmann's Encyclopedia of Industrial Chemistry, Pigments, Organic, 2012, Wiley-VCH Verlag GmbH & Co. KGaA, Weinheim and in the article by Volz, Ullmann's Encyclopedia of Industrial Chemistry, Pigments, Inorganic, 1. General, 2012, Wiley-VCH Verlag GmbH & Co. KGaA, Weinheim.

As mineral pigments that may be used in the invention, mention may be made of titanium oxide, titanium dioxide, zirconium oxide, zirconium dioxide, cerium oxide or cerium dioxide and also zinc oxide, iron oxide or chromium oxide, ferric blue, manganese violet, ultramarine blue and chromium hydrate, and mixtures thereof.

They may also be pigments having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30.

They may also be pigments having a structure that may be, for example, of silica microsphere type containing iron oxide. An example of a pigment having this structure is the product sold by Miyoshi under the reference PC Ball PC-LL-100 P, this pigment being constituted of silica microspheres containing yellow iron oxide.

Advantageously, the pigments in accordance with the invention are natural pigments or pigments of natural origin.

### Nacres

For the purposes of the present invention, the term “nacres” denotes coloured particles of any form, which may or may not be iridescent, notably produced by certain molluscs in their shell, or alternatively synthesized, and which have a colour effect via optical interference. The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles, at the surface of which are superposed at least two successive layers of metal oxides and/or of organic dyestuffs.

Examples of nacres that may also be mentioned include natural mica covered with titanium oxide, with iron oxide, with natural pigment or with bismuth oxychloride.

Among the nacres available on the market, mention may be made of the Timica, Flamenco and Duochrome nacres (based on mica) sold by the company Engelhard, the Timiron nacres sold by the company Merck, the Prestige mica-based nacres, sold by the company Eckart, and the Sunshine synthetic mica-based nacres, sold by the company Sun Chemical.

The nacres may more particularly have a yellow, pink, red, bronze, orange, brown, gold and/or coppery colour or tint.

Particles with a Metallic Tint

For the purposes of the present invention, the term “particles with a metallic tint” means any compound whose nature, size, structure and surface finish allow it to reflect the incident light, notably in a non-iridescent manner.

The particles with a metallic tint that may be used in the invention may in particular be chosen from:
particles of at least one metal and/or of at least one metal derivative;
particles including a single-material or multi-material organic or mineral substrate, at least partially coated with at least one layer with a metallic tint comprising at least one metal and/or at least one metal derivative; and
mixtures of said particles.

Among the metals that may be present in said particles, mention may be made, for example, of Ag, Au, Cu, Al, Ni, Sn, Mg, Cr, Mo, Ti, Zr, Pt, Va, Rb, W, Zn, Ge, Te and Se, and mixtures or alloys thereof. Ag, Au, Cu, Al, Zn, Ni, Mo and Cr, and mixtures or alloys thereof (for example bronzes and brasses) are preferred metals.

The term “metal derivatives” denotes compounds derived from metals, notably oxides, fluorides, chlorides and sulfides.

Illustrations of these particles that may be mentioned include aluminium particles, such as those sold under the names Starbrite 1200 EAC® by the company Siberline and Metalure® by the company Eckart and glass particles coated with a metallic layer, notably those described in JP-A-09188830, JP-A-10158450, JP-A-10158541, JP-A-07258460 and JP-A-05017710.

### Hydrophobic Treatment

The pulverulent dyestuffs as described previously may be totally or partially surface treated, with a hydrophobic agent, to make them more compatible with the oily phase of the composition of the invention, notably so that they have good wettability with oils. Thus, these treated pigments are well dispersed in the oily phase.

Hydrophobically treated pigments are notably described in EP-A-1 086 683.

The hydrophobic-treatment agent may be chosen from silicones such as methicones, dimethicones and perfluoroalkylsilanes; fatty acids, such as stearic acid; metal soaps, in particular aluminium dimyristate, the aluminium salt of hydrogenated tallow glutamate; perfluoroalkyl phosphates; polyhexafluoropropylene oxides; perfluoropolyethers; amino acids; N-acylamino acids or salts thereof; lecithin, isopropyl triisostearyl titanate, isostearyl sebacate, and mixtures thereof.

The term “alkyl” mentioned in the compounds cited above notably denotes an alkyl group containing from 1 to 30 carbon atoms and preferably containing from 5 to 16 carbon atoms. Preferably, the dyestuff is chosen from natural dyestuffs or dyestuffs of natural origin.

According to a particular embodiment, the dyes or pigments that are suitable for use in the invention are derived from extracts of animals, bacteria, fungi, algae or plants, used in their entirety or partially.

The dye(s) or pigment(s) that are suitable for use in the invention are preferentially derived from extracts of plants or plant parts such as the fruit, including citrus fruit, vegetables, trees and shrubs. Mixtures of these extracts may also be used.

Thus, the dyestuff may be introduced into a fragranced composition according to the invention in the form of a natural extract.

Such extracts may be obtained by extraction of various plant parts, for instance the root, the wood, the bark, the leaf, the flower, the fruit, the seed, the pod or the peel.

Among the plant extracts, mention may be made of extracts of rose, gardenia or Clitoria ternatea leaves or of blue pea flowers, of curcuma rhizomes, or alternatively of carrot, sweet potato or radish peelings.

Among the extracts of fruit, mention may be made of extracts of apple, extracts of grape or extracts of cocoa beans and/or pods.

Among the extracts of vegetables, mention may be made of extracts of potato, of onion peel and of beetroot.

Among the extracts of tree wood, mention may be made of extracts of pine bark and extracts of logwood.

Use may also be made of mixtures of plant extracts.

As examples of dyestuffs that are more particularly suitable for use in the invention, mention may be made of natural water-soluble dyes or water-soluble dyes of natural origin, in particular dyes derived from natural ortho-diphenol.

More particularly, the ortho-diphenols that may be used according to the invention are chosen from:
flavanols, in particular catechin and epicatechin gallate,
flavonols, in particular quercetin;
pyrans, in particular anthocyanidins, in particular cyanidin, delphinidin and petunidin, anthocyanins or anthocyans, in particular myrtillin or anthocyanosides;
ortho-hydroxybenzoates, for example gallic acid salts,
flavones, in particular luteolin,
hydroxystilbenes, for example 3,3′,4,5′-tetrahydroxystilbene, optionally oxylated (for example glucosylated),
3,4-dihydroxyphenylalanine and derivatives thereof,
2,3-dihydroxyphenylalanine and derivatives thereof,
4,5-dihydroxyphenylalanine and derivatives thereof,
dihydroxycinnamates,
ortho-polyhydroxycoumarins,
ortho-polyhydroxyisocoumarins,
ortho-polyhydroxycoumarones;
ortho-polyhydroxyisocoumarones,
ortho-polyhydroxychalcones,
ortho-polyhydroxychromones,
quinones,
hydroxyxanthones,
1,2-dihydroxybenzene and derivatives thereof,
1,2,4-trihydroxybenzene and derivatives thereof,
1,2,3-trihydroxybenzene and derivatives thereof,
2,4,5-trihydroxytoluene and derivatives thereof,
proanthocyanidins and notably the proanthocyanidins A1, A2, B1, B2, B3 and C1,
proanthocyanins,
tannic acid,
gallic acid,
ellagic acid,
neoflavanols and neoflavanones, in particular derivatives of haematoxylin, of haematin, of brazilin and of brazilein; and
mixtures of the preceding compounds.

Preferably, the ortho-diphenols that may be used according to the invention are chosen from anthocyanidins, in particular cyanidin, delphinidin and petunidin; anthocyanins or anthocyans, in particular myrtillin; proanthocyanidins and notably the proanthocyanidins A1, A2, B1, B2, B3 and C1; proanthocyanins and mixtures of the preceding compounds.

As examples of dyestuffs that are more particularly suitable for use in the invention, mention may be made of those chosen from:
pyran or pyranium dyes or pigments, in particular anthocyans and anthocyanosides, preferably extracts of red radish, extracts of sweet potato, notably of violet sweet potato; and
polyene dyes or pigments notably chosen from jasminoids, in particular those in which the coloured active agents have the following formula:

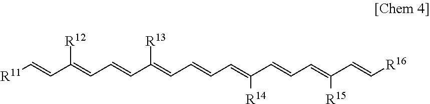

in which:
R¹¹ and R¹⁶, which may be identical or different, represent:
i) a cycloalkyl group which is optionally unsaturated and/or optionally substituted with one or more groups chosen from a) oxo, b) hydroxyl, and c) (C₁-C₆)alkyl, preferably (C₁-C₄)alkyl, in particular methyl;
ii) a cycloalkylcarbonyl group in which the cycloalkyl group is optionally unsaturated and/or optionally substituted with one or more groups a) to c) as defined previously;
iii) —C(O)—O—R¹⁷ with R¹⁷ representing a hydrogen atom or a (C₁-C₆)alkyl and preferably (C₁-C₄)alkyl group, in particular methyl;
iv) sugar, in particular glycosyl, glycosyloxy; or
v) sugar-C(O)—, and
R¹² to R¹⁵, which may be identical or different, preferably identical, represent a hydrogen atom or a (C₁-C₆)alkyl group; preferably, R¹² to R¹⁵ represent a (C₁-C₄)alkyl group, for example methyl,
and also the geometrical isomers thereof, the organic or mineral base salts thereof, and the solvates thereof such as hydrates.

As examples of dyestuffs that are suitable for use in the invention, mention may also be made of extracts of red, blue or yellow gardenia, carotenes, such as β-carotenes, paprika and curcumin, the coloured active agents of which have the following formula, or a geometrical isomer thereof, an organic or mineral base salt thereof or a solvate thereof, in particular hydrates thereof:

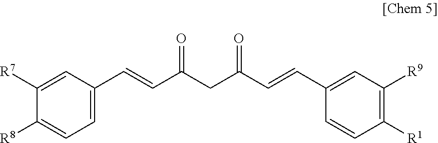

in which R⁷, R⁸, R⁹ and R¹⁰ represent, independently of each other, a hydrogen atom, a hydroxyl group or a (C₁-C₆)alkoxy and preferably (C₁-C₄)alkoxy group, in particular methoxy, it being understood that at least one of the groups R⁷ to R¹⁰ represents a hydroxyl group.

Preferably, R⁸ and R¹⁰ represent a hydroxyl group, R⁷ represents a hydrogen atom or a (C₁-C₄)alkoxy group, in particular methoxy, and R⁹ represents a hydrogen atom or a (C₁-C₄)alkoxy, in particular methoxy.

According to a preferred embodiment, the dyestuff is chosen from:
pyran or pyranium dyes or pigments, in particular anthocyans and anthocyanosides, preferably extracts of red radish, extracts of sweet potato, notably of violet sweet potato;
polyene dyes or pigments notably chosen from jasminoids, in particular those in which the coloured active agents have the following formula:

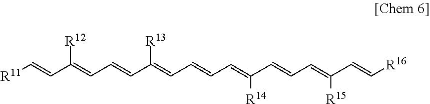

in which:
R¹¹ and R¹⁶, which may be identical or different, represent:
i) a cycloalkyl group which is optionally unsaturated and/or optionally substituted with one or more groups chosen from a) oxo, b) hydroxyl, and c) (C₁-C₆)alkyl, preferably (C₁-C₄)alkyl, in particular methyl;
ii) a cycloalkylcarbonyl group in which the cycloalkyl group is optionally unsaturated and/or optionally substituted with one or more groups a) to c) as defined above;
iii) —C(O)—O—R¹⁷ with R¹⁷ representing a hydrogen atom or a (C₁-C₆)alkyl and preferably (C₁-C₄)alkyl group, in particular methyl;
iv) sugar, in particular glycosyl, glycosyloxy; or
v) sugar-C(O)—, and
R¹² to R¹⁵, which may be identical or different, preferably identical, represent a hydrogen atom or a (C₁-C₆)alkyl group; preferably, R¹² to R¹⁵ represent a (C₁-C₄)alkyl group, for example methyl,
and also the geometrical isomers thereof, the organic or mineral base salts thereof, and the solvates thereof such as hydrates; and
   curcumin, the coloured active agents of which have the following formula:

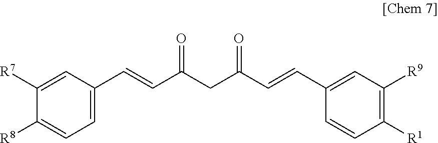

in which R⁷, R⁸, R⁹ and R¹⁰ represent, independently of each other, a hydrogen atom, a hydroxyl group or a (C₁-C₆)alkoxy and preferably (C₁-C₄)alkoxy group, in particular methoxy, it being understood that at least one of the groups R⁷ to R¹⁰ represents a hydroxyl group; preferably, R⁸ and R¹⁰ represent a hydroxyl group, R⁷ represents a hydrogen atom or a (C₁-C₄)alkoxy group, in particular methoxy, and R⁹ represents a hydrogen atom or a (C₁-C₄)alkoxy group, in particular methoxy,
and also the geometrical isomers thereof, the organic or mineral base salts thereof, and the solvates thereof such as hydrates.

As other examples of dyestuffs that are suitable for use in the invention, mention may be made of phthalocyanins and porphyrins, in particular complexed with a metal, preferably a transition metal (such as titanium, manganese, iron, cobalt, nickel, copper, zinc, notably magnesium or copper), such as chlorophyll, chlorophyllin and the metal complexes thereof, notably the transition metal complexes thereof, in particular of titanium, manganese, iron, cobalt, nickel, copper, zinc, notably magnesium or copper, preferably copper.

According to a particular embodiment, the dyestuffs of the invention are chosen from natural porphyrins and phthalocyanines, in particular those complexed with a metal, notably chlorophyllin and more particularly that complexed with copper or magnesium, preferably copper chlorophyllin. Mention may be made, for example, of chlorophyllin complexed with copper, in particular sold by Sensient under the name Natpure Col Green LC 712, and chlorophyll complexed with copper, in particular the product sold under the name Copper Chlorophyll/CI 75810 by the company Naturex).

More preferentially, a dyestuff that is suitable for use in the invention may be chosen from:
i) paprika, in particular the product sold under the name Capsanthin/Capsorubin by the company Vitiva;
ii) β-carotene, in particular the product sold under the name Beta-Carotene by the company Vitiva;
iii) extract of red radish, in particular the product sold under the name Raphanus sativus Root Extract by the company Vitiva, the colouring active agent of which is enocyanin;
iv) extract of violet sweet potato, in particular the product sold under the name Anthocyanins/Ipomoeia batatas Root Extract by the company Vitiva, which is an extract of the roots of Ipomoea batatas, Convolvulacaea;
v) yellow gardenia, in particular the product sold under the name Yellow Gardenia—Gardenia jasminoides Fruit Extract by the company Henan Zhongda Biological; vi) red gardenia, in particular the product sold under the name Gardenia Fruit Extract For Water Soluble Red by the company Henan Zhongda Biological;
vii) blue gardenia, in particular the product sold under the name Blue Gardenia—Hydrolyzed Gardenia florida Extract With Maltodextrin by the company Henan Zhongda Biological;
viii) curcumin, in particular the product sold under the name Curcuma longa Extract TBC by the company Naturex;
ix) extract of blue pea, in particular the product sold under the name Clitoria ternatea Flower Extract by the company Naturalsolution; and
x) chlorophyll or chlorophyllin, preferably chlorophyll, in particular the product sold under the name Copper Chlorophyll/CI 75810 by the company Naturex.

According to a preferred embodiment, the dyestuff is chosen from pyran dyes or pigments, polyene dyes or pigments, extracts of red, blue or yellow gardenia, carotenes, paprika, curcumin, porphyrins and mixtures thereof, in particular from paprika, β-carotene, extract of red radish, extract of violet sweet potato, yellow, red or blue gardenia, curcumin, extract of blue pea, chlorophyll, and mixtures thereof.

The dyestuffs that are suitable for use in the invention may be in the form of acid or base salts, in particular of mineral or organic acids, in particular hydrochloric acid, or mineral or organic bases, such as alkali metal hydroxides, including sodium hydroxide.

The dyestuffs that are suitable for use in the invention may be glycosylated, i.e. they may include one or more glycosyl groups.

All the enantiomers of the dyestuffs defined above may be suitable for use as dyestuff, as may the racemic mixtures.

The dyestuff, in particular obtained from natural extract(s), which is suitable for use in the invention may be in any form enabling its dissolution in the fragranced composition, in particular in aqueous, alcoholic or aqueous-alcoholic medium. It may notably be in powder or liquid form, preferably in powder form.

For obvious reasons, the amount of dyestuff(s) present in a composition according to the invention is able to vary significantly with regard to the hue or the chromatic intensity sought by its presence.

Preferably, the dyestuff(s) present in the composition according to the invention represent from 0.0001% to 20% by weight relative to the total weight of the composition(s) containing the dyestuff(s) or the extract(s) containing same.

As regards the pure dyestuff(s) b), the content in the fragrancing composition containing same is preferably between 0.0001% and 5% by weight, preferably between 0.001% and 3.0% by weight, more preferentially between 0.01% and 1.0% by weight and even more preferentially between 0.1% and 0.5% by weight of dyestuffs(s) relative to the total weight of said composition.

According to a particular embodiment of the invention, the total amount of dyestuff(s) contained in the composition ranges from 1 to 300 ppm (parts per million) and more particularly from 1 to 200 ppm.

Advantageously, a dyestuff content of less than or equal to 300 ppm in the fragranced coloured composition makes it possible to prevent the formation of marks during its application, in particular on clothing, and irrespective of the dyestuff used.

As mentioned above, the dyestuff may be introduced into the fragranced composition of the invention in the form of a natural extract.

Thus, a fragranced composition according to the invention may comprise from 0.05% to 15% by weight of natural extract comprising a dyestuff, relative to the total weight of said composition.

Preferably, a composition according to the invention comprises between 0.0001% and 20% by weight of dyestuffs(s) relative to the total weight of the composition containing the dyestuff(s) or the extract(s) containing same, preferably:
when the dyestuff(s) are pure, the content in the fragrancing composition is between 0.0001% and 5% by weight, preferably between 0.001% and 3.0% by weight, more preferentially between 0.01% and 1.0% by weight and even more preferentially between 0.1% and 0.5% by weight of dyestuffs(s) relative to the total weight of said composition;
when the dyestuff(s) are in the form of extract(s), the content of extract(s) in the composition is between 0.01% and 20% by weight, particularly between 0.1% and 10% by weight, particularly between 0.2% and 8.0% by weight, preferentially between 0.3% and 3.0% by weight, more preferentially between 0.4% and 2.0% by weight and better still between 0.5% and 1% by weight of extract(s) in said composition relative to the total weight of said composition.

### Fragrancing Substance

As mentioned previously, a fragranced composition according to the invention comprises at least one fragrancing substance.

Perfumes are compositions notably containing the starting materials described in S.

Arctander, Perfume and Flavor Chemicals (Montclair, N.J., 1969), in S. Arctander, Perfume and Flavor Materials of Natural Origin (Elizabeth, N.J., 1960) and in Flavor and Fragrance Materials—1991, Allured Publishing Co., Wheaton, III.

A fragranced composition according to the invention preferably comprises at least one fragrancing substance chosen from essential oils, perfumes and aromas of synthetic or natural origin, and mixtures thereof.

They may be natural products, such as essential oils, absolutes, resinoids, resins, concretes, and/or synthetic products, such as terpene or sesquiterpene hydrocarbons, alcohols, phenols, aldehydes, ketones, ethers, acids, esters, nitriles or peroxides, which may be saturated or unsaturated, and aliphatic or cyclic.

According to the definition given in the international standard ISO 9235 and adopted by the Commission of the European Pharmacopoeia, an essential oil is an odorous product, generally of complex composition, obtained from a botanically defined plant starting material, either by steam distillation, or by dry distillation, or by an appropriate mechanical process without heating (cold pressing). The essential oil is generally separated from the aqueous phase via a physical process which does not result in any significant change in the composition.

The choice of the method for obtaining essential oils depends mainly on the starting material: its original state and its characteristics, its intrinsic nature. The “essential oil/plant starting material” yield may be extremely variable depending on the plant: 15 ppm to more than 20%. This choice determines the characteristics of the essential oil, in particular viscosity, colour, solubility, volatility, and richness or poorness in certain constituents.

Steam entrainment corresponds to vaporization, in the presence of steam, of a sparingly water-miscible substance. The raw material is placed in contact with boiling water or steam in an alambic. The steam entrains the essential oil vapour, which is condensed in the condenser and recovered as a liquid phase in a Florentine vase (or essence jar), where the essential oil is separated from the water by settling. The aqueous distillate that remains after the steam distillation, once the separation of the essential oil has been performed, is known as the “aromatic water” or “hydrolate” or “distilled floral water”.

Production by dry distillation consists in obtaining the essential oil by distillation of woods, barks or roots, without addition of water or steam, in a closed chamber designed so that the liquid is recovered at the bottom. Cade oil is the best known example of a product obtained in this way.

The method of production by cold pressing applies only to citrus fruits (Citrus spp.) via mechanical processes at room temperature. The principle of the method is as follows: the zests are torn into pieces and the contents of the secretory sacs that have been broken are recovered by a physical process. The conventional process consists in exerting an abrasive action on the entire surface of the fruit under a stream of water. After removal of the solid waste, the essential oil is separated from the aqueous phase by centrifugation. The majority of industrial installations allow simultaneous or sequential recovery of the fruit juices and of the essential oil.

Essential oils are generally volatile and liquid at room temperature, which distinguishes them from “set” oils. They are more or less coloured and their density is generally less than that of water. They have a high refractive index and most of them deflect polarized light. They are liposoluble and soluble in the usual organic solvents, distillable with steam, and very sparingly soluble in water.

Among the essential oils that may be used according to the invention, mention may be made of those obtained from plants belonging to the following botanical families: Abietaceae or Pinaceae, for example conifers; Amaryllidaceae; Anacardiaceae; Anonaceae, for example ylang ylang; Apiaceae, for example Umbelliferae, in particular dill, angelica, coriander, sea fennel, carrot or parsley; Araceae; Aristolochiaceae; Asteraceae, for example yarrow, artemisia, camomile, helichrysum; Betulaceae; Brassicaceae; Burseraceae, for example frankincense; Caryophyllaceae; Canellaceae; Cesalpiniaceae, for example copaifera (copaiba balsam); Chenopodaceae; Cistaceae, for example rock rose; Cyperaceae; Dipterocarpaceae; Ericaceae, for example gaultheria (wintergreen); Euphorbiaceae; Fabaceae; Geraniaceae, for example geranium; Guttiferae; Hamamelidaceae; Hernandiaceae; Hypericaceae, for example St-John's wort; Iridaceae; Juglandaceae; Lamiaceae, for example thyme, oregano, monarda, savory, basil, marjorams, mints, patchouli, lavenders, sages, catnip, rosemary, hyssop, balm, rosemary; Lauraceae, for example ravensara, sweet bay, rosewood, cinnamon, litsea; Liliaceae, for example garlic; Magnoliaceae, for example magnolia; Malvaceae; Meliaceae; Monimiaceae; Moraceae, for example hemp or hop; Myricaceae; Myristicaceae, for example nutmeg; Myrtaceae, for example eucalyptus, tea tree, paperbark tree, cajuput, backhousia, clove, myrtle; Oleaceae; Piperaceae, for example pepper; Pittosporaceae; Poaceae, for example citronella grass, lemongrass, vetiver; Polygonaceae; Renonculaceae; Rosaceae, for example roses; Rubiaceae; Rutaceae: all citrus plants; Salicaceae; Santalaceae, for example sandalwood; Saxifragaceae; Schisandraceae; Styracaceae, for example benzoin; Thymelaceae, for example agarwood; Tilliaceae; Valerianaceae, for example valerian, spikenard; Verbenaceae, for example lantana, verbena; Violaceae; Zingiberaceae, for example galangal, turmeric, cardamom, ginger; Zygophyllaceae.

Mention may also be made of the essential oils extracted from flowers (lily, lavender, rose, jasmine, ylang ylang, neroli), from stems and leaves (patchouli, geranium, petitgrain), from fruit (coriander, aniseed, cumin, juniper), from fruit peel (bergamot, lemon, orange), from roots (angelica, celery, cardamom, iris, sweet flag, ginger), from wood (pinewood, sandalwood, gaiac wood, rose of cedar, camphor), from grasses and gramineae (tarragon, rosemary, basil, lemongrass, sage, thyme), from needles and branches (spruce, fir, pine, dwarf pine) and from resins and balms (galbanum, elemi, benzoin, myrrh, olibanum, opopanax).

Examples of fragrancing substances are notably: geraniol, geranyl acetate, farnesol, borneol, bornyl acetate, linolool, linalyl acetate, linalyl propionate, linalyl butyrate, tetrahydrolinolool, citronellol, citronellyl acetate, citronellyl formate, citronellyl propionate, dihydromyrcenol, dihydromyrcenyl acetate, tetrahydromyrcenol, terpineol, terpinyl acetate, nopol, nopyl acetate, nerol, neryl acetate, 2-phenylethanol, 2-phenylethyl acetate, benzyl alcohol, benzyl acetate, benzyl salicylate, styrallyl acetate, benzyl benzoate, amyl salicylate, dimethylbenzylcarbinol, trichloromethylphenylcarbinyl acetate, p-tert-butylcyclohexyl acetate, isononyl acetate, vetiveryl acetate, vetiverol, α-hexylcinnamaldehyde, 2-methyl-3-(p-tert-butylphenyl)propanal, 2-methyl-3-(p-isopropylphenyl)propanal, 3-(p-tert-butylphenyl)propanal, 2,4-dimethylcyclohex-3-enylcarboxaldehyde, tricyclodecenyl acetate, tricyclodecenyl propionate, 4-(4-hydroxy-4-methylpentyl)-3-cyclohexenecarboxaldehyde, 4-(4-methyl-3-pentenyl)-3-cyclohexenecarboxaldehyde, 4-acetoxy-3-pentyltetrahydropyran, 3-carboxymethyl-2-pentylcyclopentane, 2-n-4-heptylcyclopentanone, 3-methyl-2-pentyl-2-cyclopentenone, menthone, carvone, tagetone, geranylacetone, n-decanal, n-dodecanal, 9-decen-1-ol, phenoxyethyl isobutyrate, phenylacetaldehyde dimethyl acetal, phenylacetaldehyde diethyl acetal, geranonitrile, citronellonitrile, cedryl acetate, 3-isocamphylcyclohexanol, cedryl methyl ether, isolongifolanone, aubepinonitrile, aubepine, heliotropin, coumarin, eugenol, vanillin, diphenyl ether, citral, citronellal, hydroxycitronellal, damascone, ionones, methylionones, isomethylionones, solanone, irones, cis-3-hexenol and esters thereof, musk-indans, musk-tetralins, musk-isochromans, macrocyclic ketones, musk-macrolactones, aliphatic musks and ethylene brassylate, and mixtures thereof.

A fragranced composition according to the invention preferably comprises a fragrancing substance chosen from essential oils, perfumes and aromas of synthetic or natural origin, and mixtures thereof.

According to a preferred embodiment of the invention, a mixture of different fragrancing substances that generate in common a note that is pleasant to the user is used.

Thus, according to a preferred embodiment, the fragranced composition comprises at least 1% by weight of a mixture of fragrancing substances, in particular of at least two different fragrancing substances, relative to the total weight of the composition, and preferably at least three different fragrancing substances.

The fragrancing substances will preferably be chosen such that they produce notes (head, heart and base) in the following families: citrine, aromatic, floral notes, in particular pink flowers and white flowers, spicy, woody, gourmand, chypre, fougere, leathery, musk.

For obvious reasons, the amount of fragrancing substance(s) present in a composition according to the invention is able to vary significantly with regard to the odour or the odour intensity sought by its presence.

By way of illustration, a fragranced composition according to the invention may comprise from 0.0001% to 10% by weight, particularly from 0.001% to 8.0% by weight, more particularly from 0.01% to 5.0% by weight and preferentially from 0.1% to 1.0% by weight of fragrancing substance(s), relative to the total weight of said composition.

As mentioned above, the fragrancing substances may be introduced into a fragranced composition in accordance with the invention in the form of a perfume concentrate.

The perfume concentrate may be a concrete or an absolute, preferably an absolute.

Thus, a fragranced composition according to the invention particularly comprises from 1.0% to 50% by weight of perfume concentrate, more particularly from 2.0% to 40% by weight, preferentially from 5.0% to 30% by weight and more preferentially from 10% to 20% by weight, for example 15% by weight, relative to the total weight of the composition.

### Composition

A fragranced composition according to the invention may be in any galenical form conventionally used for topical application, and notably in the form of an aqueous, alcoholic or aqueous-alcoholic solution or suspension, an oily solution or a solution or dispersion of the lotion or serum type, an emulsion of liquid or semi-liquid consistency of the milk type, obtained by dispersing a fatty phase in an aqueous phase (O/W) or, conversely, (W/O), a suspension or emulsion of soft consistency of (O/W) or (W/O) cream type, or an aqueous or anhydrous gel, an ointment, or any other cosmetic form.

According to a preferred embodiment, a fragranced composition according to the invention is of oil-in-water type, and is preferably in the form of an oil-in-water emulsion.

Preferably, a fragranced composition according to the invention is an aqueous composition, i.e. a composition comprising water as solvent, an alcoholic composition, i.e. a composition comprising at least one alcohol as solvent(s), or an aqueous-alcoholic composition, i.e. a composition comprising a mixture of water and of alcohol as solvents.

According to a preferred embodiment, a fragranced composition according to the invention is an aqueous, alcoholic or aqueous-alcoholic composition; preferably, the composition comprises as solvent(s) water, (bio)ethanol, pentylene glycol, or a mixture of at least two of these compounds.

### Alcohols

Preferably, a fragranced composition according to the invention is an aqueous composition, i.e. a composition comprising water as solvent, an alcoholic composition, i.e. a composition comprising at least one alcohol as solvent(s), or an aqueous-alcoholic composition, i.e. a composition comprising a mixture of water and of alcohol as solvents.

According to a preferred embodiment, a fragranced composition according to the invention is an aqueous, alcoholic or aqueous-alcoholic composition; preferably, the composition comprises as solvent(s) water, (bio)ethanol, pentylene glycol, or a mixture of at least two of these compounds.

Preferably, the composition according to the invention is aqueous-alcoholic.

The alcohols that are most particularly suitable for use in the invention are chosen from i) monoalcohols containing from 2 to 6 carbon atoms such as ethanol and isopropanol, and ii) glycols containing from 2 to 8 carbon atoms such as ethylene glycol, propylene glycol, 1,3-butylene glycol and dipropylene glycol.

Preferably, the alcohols are of natural origin. In particular, mention may be made of bioethanol.

In particular, a fragranced composition according to the invention comprises as solvent(s) water, (bio)ethanol, pentylene glycol, or a mixture of at least two of these compounds.

Preferably, the composition of the invention comprises a content of alcohols, notably of (bio)ethanol, of between 0.5% and 99.5% by weight, particularly between 5% and 97% by weight, more particularly between 15% and 95% by weight, better still between 25% and 93%, preferably between 40% and 92% by weight, more preferentially between 50% and 90% by weight, even more preferentially between 60% and 88% by weight and better still between 70% and 85% by weight, relative to the total weight of the composition.

### Water-Soluble Solvents

A composition according to the invention is preferably aqueous or aqueous-alcoholic.

An aqueous or aqueous-alcoholic fragranced composition according to the invention may also comprise one or more water-soluble solvents different from the alcohols defined above.

In the present invention, the term “water-soluble solvent” denotes a compound that is liquid at room temperature and water-miscible (miscibility with water of greater than 50% by weight at a temperature ranging from 20° C. to 25° C. and atmospheric pressure).

The water-soluble solvents that may be used in the composition of the invention may also be volatile. Among the water-soluble solvents that may be used in the composition in accordance with the invention, mention may be made notably of C₃ and C₄ ketones and C₂-C₄ aldehydes.

Alternatively, a fragranced composition according to the invention may be anhydrous, i.e. it comprises less than 2% by weight of water, preferably less than 1% by weight of water, preferentially less than 0.5% by weight of water, or even is totally free of water.

### Polyols

A fragranced composition according to the invention may also comprise at least one C₂-C₃₂ polyol. For the purposes of the present invention, the term “polyol” should be understood as meaning any organic molecule including at least three free hydroxyl (—OH) groups. Preferably, a polyol in accordance with the present invention is present in liquid form at room temperature. A polyol that is suitable for use in the invention may be a compound of linear, branched or cyclic, saturated or unsaturated alkyl type, bearing on the alkyl chain at least three —OH functions and in particular at least four —OH functions.

The polyols that are advantageously suitable for formulating a composition according to the present invention are those notably containing from 2 to 32 carbon atoms and preferably 3 to 16 carbon atoms.

Preferably, the polyol may be chosen, for example, from pentaerythritol, trimethylolpropane, glycerol, polyglycerols, in particular glycerol oligomers, preferably diglycerol, and mixtures thereof.

### Fatty Substance

A fragranced composition according to the invention may comprise one or more fatty substances that are liquid at room temperature and/or a fatty substance that is solid at room temperature such as waxes, pasty fatty substances, gums and mixtures thereof. For the purposes of the invention, the term “room temperature” means a temperature equal to 25° C. Preferably, the fatty substances that are suitable for use in the present invention are liquid at room temperature and at atmospheric pressure.

The oils may be polar or non-polar, of different chemical nature, alone or as a mixture.

In particular, polar oils that may be mentioned include:
hydrocarbon-based plant oils with a high content of triglycerides consisting of fatty acid esters of glycerol, for example like wheat germ oil, corn oil, sunflower oil, shea oil, castor oil and sweet almond oil;
synthetic esters and ethers;
saturated C₁₂ to C₂₆ fatty alcohols;
and mixtures thereof.

Moreover, the non-polar oils may be chosen in particular from silicone oils such as volatile or non-volatile, linear or cyclic polydimethylsiloxanes, which are liquid at room temperature (20° C.±5° C.) and atmospheric pressure, and linear or branched hydrocarbons of synthetic or mineral origin.

Examples of solid fatty substances that may be mentioned include waxes, notably hydrocarbon-based waxes or silicone waxes, of natural or synthetic origin, insofar as they are compatible with the other components of the composition, and insofar as they do not impair the properties, notably the olfactory and colour properties, of the fragranced composition.

Preferably, the fatty substances are plant oils. Plant oils that may be mentioned include sunflower oil, virgin olive oil, refined sesame oil, etc.

### Surfactants

A fragranced composition according to the invention may also comprise one or more anionic, cationic, nonionic, zwitterionic or amphoteric surfactants.

Nonionic surfactants that may be mentioned most particularly include fatty acid esters of sugars, notably those chosen from the group comprising esters or mixtures of esters of C₈-C₂₂ fatty acid and of sucrose, of maltose, of glucose or of fructose, and esters or mixtures of esters of C₁₄-C₂₂ fatty acid and of methylglucose. Examples of esters or mixtures of esters of fatty acid and of sucrose, maltose, glucose or fructose that may be mentioned include sucrose monostearate, sucrose distearate and sucrose tristearate, and mixtures thereof, and an example of esters or mixtures of esters of fatty acid and of methylglucose that may be mentioned is methylglucose polyglyceryl-3 distearate, sold by the company Goldschmidt under the name Tego-Care 450.

In addition, the fragranced composition according to the invention may comprise one or more co-emulsifiers. This or these co-emulsifiers may be chosen, for example, from C₁₆-C₂₂ fatty alcohols or esters of C₃-C₆ polyols with C₁₄-C₂₂ fatty acids, and mixtures thereof. Examples of co-emulsifiers that may be mentioned include cetyl alcohol, stearyl alcohol, cetearyl alcohol (mixture of cetyl alcohol and of stearyl alcohol), glyceryl stearate, and mixtures thereof, and preferably cetyl alcohol.

A fragranced composition may comprise from 0.01% to 10% by weight, more particularly from 0.1% to 5% by weight and preferentially from 1% to 3% by weight of surfactant(s) relative to the total weight of the fragranced composition.

### Preserving Agents

A fragranced composition according to the invention may also comprise at least one preserving agent.

The term “preservative” or “preserving agent” means any cosmetically or pharmaceutically acceptable compound which can prevent microbial growth (or the growth of microorganisms) which may take place in cosmetic or pharmaceutical compositions, from their conception, passing through their storage, up to the time of their conventional use by consumers. Preserving agents that may notably be mentioned include the preserving agents described in Cosmetics, Kirk-Othmer Encyclopedia of Chemical Technology. John Wiley & Sons, Inc., Martin M. Rieger; 5.2 preservatives & table 3, Apr. 12, 2000, https://doi.org/10.1002/0471238961.0315191318090507.a01.

According to a particular embodiment, the preserving agent(s) are chosen from organic preserving agents bearing an aromatic group.

A fragranced composition according to the invention may also comprise at least one preserving agent, notably chosen from i) benzene-based carboxylic acids optionally substituted on the phenyl group with one or more groups chosen from hydroxyl, (C₁-C₁₀)alkyl and (C₁-C₁₀)alkylcarbonyl, and also the base salts thereof, notably of alkali metals and alkaline-earth metals, ii) hydroxybenzoic acid esters optionally substituted on the phenyl group with one or more groups chosen from (C₁-C₁₀)alkyl such as parabens, notably methylparaben and ethylparaben and propylparaben, and iii) aromatic alcohols. Preferably, the preserving agent(s) other than a) are chosen from i) and in particular benzoic acid [CAS No. 65-85-0] and also the base salts thereof, notably of alkali metals and alkaline-earth metals; and also salicylic acid optionally substituted with a (C₁-C₅)alkylcarbonyl group, preferably salicylic acid. The preserving agent(s) are notably chosen from aryl(C₁-C₆)alkanols and aryloxy(C₁-C₆)alkanols, such as phenylethanol, parabens, such as propylparaben, methylparaben, butylparaben, ethylparaben and isobutylparaben, benzyl alcohol and/or chlorophenesin.

### Alkaline Agents

A fragranced composition according to the invention may also comprise at least one organic or mineral alkaline agent, for example an amine, in particular a (C₂-C₆)alkanolamine, in particular 2-amino-2-methyl-1-propanol, or a mineral alkaline agent, for instance alkali metal or alkaline-earth metal hydroxides, such as sodium hydroxide.

### Chelating Agents

According to a particular embodiment of the invention, the composition comprises one or more chelating agents.

The term “chelating agent” means a ligand or chelating agent which makes it possible to form a metal complex with a chelated cation or metal atom. Generally, the chelating agent is electron-donating and the chelated species is electron-deficient. A “chelate” differs from a simple “complex” in that the metal cation is attached to the chelating ligand or chelating agent via at least two coordination bonds. The metal is thus “gripped” between the chemical functions of the ligand. The number of metal-ligand bonds of a ligand molecule defines the “denticity”: such coordinates or ligands are referred to as being bidentate, tridentate or tetradentate. The central atom is bonded to the neighbouring atoms via at least two bonds, forming a ring structure, a chelate ring. The chelate rings that are the most stable are 5- and 6-membered chelate rings. By means of this effect, chelates are more stable complexes than the complexes of monodentate ligands including the same chemical functions.

In particular, chelating agents are chosen from aminocarboxylic acids, for example ethylenediaminetetraacetic acid (EDTA), aminotriacetic acid, diethylenetriaminepentaacetic acid, and the salts thereof, in particular the alkaline salts, for instance disodium EDTA or dipotassium EDTA, aminopolycarboxylic acids, for instance ethylenediaminedisuccinic acid (EDDS), and salts thereof, in particular alkaline salts, or alternatively one or more phosphate-based chelating agents such as alkali metal metaphosphates such as sodium metaphosphate, alkali metal hexametaphosphates such as sodium hexametaphosphate, tetra-alkaline pyrophosphates such as tetrapotassium pyrophosphate, acids containing phosphorus, such as phytic acid, and salts thereof, in particular alkali metal or alkaline-earth metal salts, such as ethylenediaminetetramethylenephosphonic acid salts, alkali metal phytates such as sodium phytate or potassium phytate, and mixtures thereof, preferably chosen from EDTA as defined previously and alkali metal phytates such as sodium phytate.

Metal salts According to a particular embodiment of the invention, the composition of the invention comprises one or more metal salts, metal complexes, metal oxides, metal oxoanions, and the hydrates and supported forms thereof.

Preferably, a composition according to the invention comprises one or more metal salts.

More particularly, the metal(s) are of oxidation state I or II, more particularly II, preferably chosen from 1) Cu, 2) Fe, 3) Zn, 4) Mg and 5) Mn.

The term “metal salt” means a compound other than alloys, i.e. the salt is formed from a metal combined with certain non-metallic elements.

The formation of metal salts derives from oxidative attack. The metal is oxidized to a cationic species and then combines with an anionic species to give a salt. This formation takes place by applying redox principles and the redox reaction (chemical reaction during which an electron transfer takes place in which the atom that captures the electrons is known as the “oxidizing agent”; the atom that yields the electrons is the “reducing agent”); or via chemical exchange reactions between one salt and another salt or an acid, in the presence or absence of atmospheric oxygen. These reactions are known to those skilled in the art.

Preferentially, the salts according to the invention are soluble in water to a proportion of at least 0.0001 g/L.

The metal salts according to the invention may be introduced in solid form into the compositions or may originate from a natural, mineral or spring water that is rich in these ions, or alternatively from seawater (notably the Dead Sea). They may also originate from mineral compounds, such as plant extracts containing same (cf., for example, FR 2 814 943). The term “metal complex” or “coordination compounds” mean systems in which the metal ion, the central atom, is chemically bonded to one or more electron donors (ligands). A ligand comprising various coordinating groups (capable of coordinating with a metal) gives metal compounds corresponding to coordination sphere principles with a given number of electrons (internal complexes or chelates), as described in Ullmann's Encyclopedia of Industrial Chemistry, “Metal complex dyes”, 2005, pages 1-42.

More particularly, the term “metal complex” means:
i) metallic dyes or “metal-complex dyes”, which are complexed dyes derived from azo, azomethine, hydrazono or formazan dyes (free, bidentate, tridentate, tetradentate) such as those described in Ullmann's Encyclopedia of Industrial Chemistry, “Metal complex dyes”, 2005, pages 1-42, which preferentially comprise Cu and Mg;
ii) compounds of the “aza[18]annulene” type, also known as “(metallo)porphyrins” and “phthalocyanins” which contain 4 and 8 nitrogen atoms, respectively, included in the parameter of the macrocycle, as described in the book “Color Chemistry”, H. Zollinger, 3rd Ed., Wiley-VCH, 2003, chapter 5, Aza[18]annulenes, pages 123-160. The metal ion is then at the centre of said macrocycle bonded by coordination with two hydrogen atoms to the nitrogen atoms of pyrroles, the metal also possibly being stabilized with one or more bidentate or non-bidentate ligands, the metal ion preferentially being Mg²⁺ or Cu²⁺.
the metal complex is particularly:
   a “metalloporphyrin” formed from a backbone bearing 4 pyrrole groups that are connected at their α,α′ position via 4 methine groups and contain 16 sp² hybridized atoms, complexing a metal such as Cu or Mg, or
   a “chlorine” (corresponding to a porphyrin in which an external C═C double bond of a pyrrole group has been reduced) complexed to a metal, preferentially Mg²⁺ such as the chlorophyll chromophore: chlorophyllin;
   “phthalocyanins”, which are tetraaza analogues of tetrabenzoporphyrins, such as Monastral Fast Blue B (C.I. Pigment Blue 15); Monastral Fast Blue G (C.I. Pigment Blue 16) (see “Color Chemistry”, H. Zollinger, 3rd Ed., Wiley-VCH, 2003, chapter 5, Aza[18]annulenes, page 140); the sulfonyl derivatives Sirius Light Turquoise Blue G (C.I. Direct Blue 86, copper phthalocyanin tetrasulfonic acid) and “phthalocyanins” as described in Ullmann's Encyclopedia of Industrial Chemistry, “Phthalocyanins”, 2005, pages 1-34, which comprise Cu and Si.

The term “metal oxide(s)” means compounds of general formula AₓO_{y} with A representing a metal element, x being greater than or equal to 1 and less than or equal to 4, and y being greater than or equal to 1 and less than or equal to 12.

The term “metal oxoanion(s)” means compounds of general formula Z_{z}AₓO_{y} with A representing a metal element, Z representing an alkali metal such as Li, Na or K or a hydrogen atom or an ammonium ion, z being greater than or equal to 1 and less than or equal to 6, x being greater than or equal to 1 and less than or equal to 4, and y being greater than or equal to 1 and less than or equal to 12.

The term “supported form(s)” means the forms in which the metal derivative b) is impregnated onto a material known as a “support”. The optional supports for these metal derivatives may be chosen from charcoal, silica, alumina, optionally charged polymers comprising counter-anions or counter-cations (counter-cation or counter-anion of the metallic species). By way of example, the polymers may be polyethylene glycol (PEG).

According to one embodiment of the invention, the metal salt(s) are chosen from the (poly)hydroxyalkyl(C₁-C₁₆)carboxylates of metals of oxidation state II, notably Fe II, Cu II, Zn II, Mg II and Mn II (poly)hydroxyalkyl(C₁-C₁₆)carboxylates. In particular, the metal salt(s) are chosen from Cu gluconate, Fe gluconate, Zn gluconate or Mg gluconate, gluconates of metals of oxidation state II such as Fe, Cu, Zn or Mg gluconates, and lactates of metals of oxidation state II such as Fe, Cu, Zn or Mg lactates. Preferably, the metal salts are Fe, Cu, Mg or Zn gluconate, preferably Fe and Cu gluconate.

### Aqueous pH Buffers

A fragranced composition according to the invention may also comprise at least one aqueous pH buffer, namely at least one compound which makes it possible to maintain a constant pH in aqueous solution.

The aqueous pH buffer(s) that are particularly suitable for use in the invention may be chosen from benzoic acid, maleic acid, fumaric acid, succinic acid and the salts thereof, in particular the alkali metal or alkaline-earth metal salts, for example sodium benzoate.

### Additives

A fragranced composition according to the invention may also comprise any additive usually used in the field of fragrances, for instance emollients or softeners, in particular sweet almond oil, apricot kernel oil, moisturizers, in particular glycerol, calmatives, in particular α-bisabolol, allantoin and Aloe vera; vitamins and derivatives thereof, essential fatty acids, insect repellents, propellants, peptizers, fillers, cosolvents, UV-screening agents, stabilizers or preserving agents other than the compounds of formula (I) as defined according to the invention, and mixtures thereof.

It is a matter of routine practice for those skilled in the art to adjust the nature and the amount of the additives present in the compositions in accordance with the invention such that the desired cosmetic properties thereof are not thereby affected.

When they are present in the composition of the invention, these additives may be present in an amount ranging from 0.001% to 10% by weight and better still from 0.01% to 5% by weight relative to the total weight of the composition.

### Preparation of the Composition

A composition according to the invention may be obtained via any method known to those skilled in the art for the formulation of coloured fragranced compositions.

In particular, a fragranced composition according to the invention may be obtained by simple mixing, at room temperature, namely at a temperature ranging from 20° C. to 25° C., of the compounds constituting the composition.

According to a particular embodiment, a fragranced composition according to the invention results from mixing between a colouring solution, on the one hand, and a fragranced solution comprising the compound of formula (I), on the other hand.

Said colouring solution thus comprises at least one dyestuff, in particular as defined above, in a physiologically acceptable medium, in particular an aqueous or aqueous-alcoholic medium.

Said fragranced solution comprises, for its part, at least one fragrancing substance, in particular as defined above, and at least one compound of formula (I) as defined above, in a physiologically acceptable medium, in particular an aqueous or aqueous-alcoholic medium. Said fragranced solution may itself result from mixing between an intermediate fragranced solution, comprising at least one fragrancing substance, in particular as defined above, in a physiologically acceptable medium, in particular in aqueous or aqueous-alcoholic medium, and a compound of formula (I) as defined above, directly or which is in solution in a physiologically acceptable medium, in particular an aqueous or aqueous-alcoholic medium. Alternatively, the compound(s) of formula (I) may be introduced, directly or in solution, into a fragranced composition, also comprising at least one fragrancing substance and at least one dyestuff, in a physiologically acceptable medium.

It is within the competence of a person skilled in the art to adapt the conditions for preparing a fragranced composition according to the invention.

### Intended Use of the Composition

The invention applies not only to fragrancing products but also to cosmetic products for caring for or treating keratin materials, in particular the skin, including the scalp, and the lips, containing an odorous substance.

A composition according to the invention may thus constitute a cosmetic composition for fragrancing, caring for or treating keratin materials, and may notably be in the form of an eau fraiche, eau de toilette, eau de parfum, aftershave lotion, care lotion, or silicone or hydrosilicone care oil. It may also be in the form of a fragranced two-phase lotion (eau de toilette phase/hydrocarbon-based oil and/or silicone oil phase), a body milk or a shampoo.

The compositions according to the invention may be packaged in the form of bottles.

A fragrancing composition according to the invention may be diffused by various systems, such as sprays, aerosols or piezoelectric devices.

They may also be applied in the form of fine particles by means of mechanical pressurization or propellant gas devices. The devices in accordance with the invention are well known to those skilled in the art and comprise pump-bottles or “sprays”, aerosol containers comprising a propellant and aerosol pumps using compressed air as propellant. These devices are notably described in U.S. Pat. Nos. 4,077,441 and 4,850,517.

The compositions conditioned as aerosols in accordance with the invention generally contain conventional propellants, for instance dimethyl ether, isobutane, n-butane or propane.

According to a particular form of the invention, the compositions according to the invention are lotions and preferably have a viscosity ranging from 10 to 120 DU, more preferentially from 30 to 120 DU and even more preferentially from 40 to 80 DU; the viscosity being measured using a Rheomat TVe-05 viscometer, at 25° C., rotation speed 200 rpm, spindle No. 1, 10 minutes.

These low viscosities allow the compositions of the invention to be packaged using mechanical pressurization or propellant gas devices so as to be applied in the form of fine particles (vaporization).

A fragranced composition according to the invention may also be in the form of a body milk or in the form of a shampoo.

The invention is illustrated in greater detail by the examples presented below. Unless otherwise indicated, the amounts shown are expressed as mass percentages.

### EXAMPLES

### Measurement and Evaluation Methods

The stability of the compositions is evaluated by observation of the change in colour over time, visually and using a spectrocolorimeter (L*, a*, b*) Konica Minolta CM 3600A, and by olfactory evaluation of the change in the notes of the fragranced composition.

In particular, the compositions are poured into 50 mL borosilicate glass bottles and are then observed after storage for 2 months, at atmospheric pressure and under various conditions:
a) in the refrigerator, at a temperature of 4° C.;
b) at room temperature, namely between 20° C. and 25° C., protected from light;
c) in an oven, at a temperature of 37° C.;
d) in an oven, at a temperature of 45° C.; or
e) in natural light and at room temperature, namely between 20° C. and 25° C., and then:
e.1) 16 hours in an Atlas brand SunTest CPS+ ageing device, equipped with a xenon lamp, irradiating light source whose spectral distribution is close to that of sunlight, delivering an energy of 765 W/m², simulating exposure to neon lights in a shop; or
e.2) 2 weeks in an oven at a temperature of 55° C., simulating an extreme condition for the perfume.

Storage of the compositions for two months at 45° C. simulates accelerated ageing of the product corresponding to a shelf life of 3 years.

The intensity difference DL between a reference composition and a composition tested after storage under the conditions mentioned above may be calculated from the L*a*b values according to the following formula:

$$DL = {L}_{2}^{*} - {L}_{1}^{*} \qquad [ Math 1 ]$$

in which L₁* is the intensity of the colour of the reference composition and L₂* is the intensity of the colour of the composition tested after storage.

### Example 1

### Preparation of the Dye Solutions

The dye solutions C1 to C10 are prepared using the weight proportions described in detail in Table 1 below.

The values are expressed as weight percentages relative to the total weight of the dye solution.

TABLE 1

| | Compound | | | | | | | | | |
|---|---|---|---|---|---|---|---|---|---|---|
| | C1 | C2 | C3 | C4 | C5 | C6 | C7 | C8 | C9 | C10 |
| Absolute ethanol | 99.7 | 99.0 | — | — | — | — | — | 99.7 | 99.5 | 99 |
| Ethanol | — | — | 63.0 | 63.0 | — | — | — | — | — | — |
| Distilled water | — | — | 27.0 | 27.0 | 99.9 | 68.0 | 49.0 | — | — | — |
| Pentylene glycol | — | — | — | — | — | 30.0 | 50.0 | — | — | — |
| Capsanthin/capsorubin (Vitiva) | 0.3 | — | — | — | — | — | — | — | — | — |
| Beta-carotene (Vitiva) | — | 1.0 | — | — | — | — | — | — | — | — |
| Raphanus sativus root extract (Vitiva) | — | — | 10 | — | — | — | — | — | — | — |
| Anthocyanins/Ipomoeia batatas root extract (Vitiva) | — | — | — | 10 | — | — | — | — | — | — |
| Yellow gardenia - Gardenia jasminoides fruit extract | — | — | — | — | 0.1 | — | — | — | — | — |
| (Henan Zhongda Biological) | | | | | | | | | | |
| Gardenia fruit extract for water-soluble red (Henan | — | — | — | — | — | 2.0 | — | — | — | — |
| Zhongda Biological) | | | | | | | | | | |
| Blue gardenia - Hydrolysed Gardenia florida | — | — | — | — | — | — | 1.0 | — | — | — |
| extract with maltodextrin (Henan Zhongda Biological) | | | | | | | | | | |
| Curcuma longa extract TBC (Naturex) | — | — | — | — | — | — | — | 0.3 | — | — |
| Clitoria ternatea flower extract (Naturalsolution) | — | — | — | — | — | — | — | — | 0.5 | — |
| Copper chlorophyll (Naturex) | — | — | — | — | — | — | — | — | — | 1.0 |

### Protocol for Preparing the Dye Solutions

The colouring compositions are prepared by dissolving the dyes in the appropriate solvent or mixture of solvents.

In particular, for 100 g of colouring solution, the required amount of dye is weighed out in a beaker. The amount of solvents (ethanol, water and/or propylene glycol) is then added and the mixture is left stirring by magnetic stirring for 20 minutes.

### Example 2

### Preparation of the Coloured Fragranced Compositions

A fragranced composition P1 according to the invention and a fragranced composition P2 outside the invention are prepared using the weight proportions as detailed in Table 2 below.

The values are expressed as weight percentages relative to the total weight of the fragranced composition.

TABLE 2

| | | P2 |
|---|---|---|
| | P1 | Outside the |
| Compound | Invention | invention |
| Ethanol | 78 | 78 |
| Water | qs 100 | qs 100 |
| Ferulic acid (Oryza ferulix - | 0.5 | — |
| of natural origin) | | |
| Perfume concentrate⁽*⁾⁽**⁾ | 15.0 | 15.0 |
| Colouring solution C1 to C10 | 0.05 | 0.05 |

⁽*⁾Perfume of the floral woody musk type characterized by:
its head notes of peach, coriander, plum and green notes,
its heart notes of peony, lily, freesia, lily of the valley, jasmine, ylang-ylang, rose and coffee, and
its base notes of tonka bean, musk, vanilla, sandalwood and cedarwood; and/or
⁽**⁾Perfume of the citrine type characterized by:
its head notes of lime, citron, mint and eucalyptus, and
its heart notes of coconut.

### Protocol for Preparing the Coloured Fragranced Compositions

15 g of perfume concentrate are weighed out in a beaker.

950 ethanol is added to the perfume concentrate, and water is then added.

For composition P1, 0.5 g of ferulic acid is added and the mixture is then left under magnetic stirring for 10 minutes, so as to obtain a fragranced solution. 0.05 g of the colouring solution is then added using a pipette.

Composition P2 is prepared according to the same protocol, except for the addition of ferulic acid.

### Example 3

### Stability Tests

The stability of the coloured fragranced compositions is evaluated visually according to the protocol detailed above.

The change in colour of the compositions according to the invention (P1) and outside the invention (P2), as a function of the colouring solution used, is observed visually after 16 hours in the SunTest ageing device. The results are detailed in Table 3 below.

TABLE 3

| Colouring | Composition P2 | Composition P1 |
|---|---|---|
| solution | (Outside the invention) | (Invention) |
| C1 | Clear - Colourless | Clear - Orange |
| C2 | Clear - Colourless | Clear - Pale yellow |
| C3 | Clear - Colourless | Clear - Orange-red |
| C4 | Clear - Colourless | Clear - Pale pink |
| C5 | Clear - Colourless | Clear - Pale yellow |
| C6 | Clear - Colourless | Clear - Pink |
| C7 | Clear - Colourless | Clear - Pale blue |
| C8 | Clear - Colourless | Clear - Pale yellow |
| C9 | Clear - Colourless | Clear - Pale blue |
| C10 | Clear - Colourless | Clear - Pale green |

The odour of the compositions outside the invention is degraded, whereas no degradation of the odour is observed for the compositions according to the invention.

The study was performed with various perfume concentrates from different olfactory families, similar results being obtained for each perfume concentrate.

Fragranced compositions comprising the colouring solution C₇ were also evaluated after storage for 2 months at 37° C. and after storage for 2 months at 45° C.

A slight change in the colour of the fragranced composition P1 according to the invention is observed after storage for 2 months at 37° C. The blue colour is still visible after storage for 2 months at 45° C.

Composition P2 outside the invention, not comprising ferulic acid, becomes colourless after storage for only 1 month at 37° C.

Thus, the composition according to the invention conserves its colour for a significantly longer time. Moreover, its odour does not degrade over time. In addition, the compound of formula (I) used in the composition according to the invention is of natural origin.

In conclusion, the fragranced composition according to the invention is more stable over time than a composition not in accordance with the invention.

### Example 4

### Comparative Tests

The fragranced composition P3 outside the invention, not comprising ferulic acid but comprising ethylhexyl methoxycinnamate, was prepared from the weight proportions as detailed in Table 4 below.

The values are expressed as weight percentages relative to the total weight of the fragranced composition.

TABLE 4

| | P3 |
|---|---|
| | Outside the |
| Compound | invention |
| Ethanol | 78 |
| Water | qs 100 |
| Ethylhexyl methoxycinnamate (Parsol MCX) | 0.75 |
| Perfume concentrate ⁽*⁾⁽**⁾ | 15.0 |
| Colouring solution C7 | 0.05 |
| Colouring solution C7 | 0.05 |

⁽*⁾ Perfume of the floral woody musk type characterized by:
its head notes of peach, coriander, plum and green notes,
its heart notes of peony, lily, freesia, lily of the valley, jasmine, ylang-ylang, rose and coffee, and
its base notes of tonka bean, musk, vanilla, sandalwood and cedarwood; and/or
⁽**⁾ Perfume of the citrine type characterized by:
its head notes of lime, citron, mint and eucalyptus, and
its heart notes of coconut.

The fragranced composition P3 is prepared according to the preparation protocol detailed in Example 2 above for composition P1, except that the 0.5 g of ferulic acid is replaced with 0.75 g of ethylhexyl methoxycinnamate.

The molar amount of ethylhexyl methoxycinnamate in composition P3 is identical to the molar amount of ferulic acid in composition P1.

The stability of the fragranced composition is evaluated visually and using a spectrophotometer according to the protocol detailed above.

The change in colour of the compositions according to the invention (P1) and outside the invention (P2 and P3) is observed visually after 16 hours in the SunTest ageing device. The results are detailed in Table 5 below.

As a reference, the colour of the colouring solution C7 was also evaluated before ageing. Thus, the intensity difference between the reference and compositions P1, P2 and P3 after storage and ageing was able to be calculated (DL).

TABLE 5

| | | Intensity | |
|---|---|---|---|
| Composition | Visual | (L) | DL |
| Colouring solution C7 (reference) | Clear - Blue | L 94.25 | — |
| P1 (invention) | Clear - Pale blue | L 95.95 | 1.7 |
| P2 (outside the invention) | Clear - Colourless | L 99.54 | 5.29 |
| P3 (outside the invention) | Clear - Pale green | L 96.93 | 2.68 |

The blue colour is still visible for the fragranced composition P1 according to the invention after storage for 2 months in natural light and at room temperature, and then for 16 hours in the SunTest device. This is confirmed by a low intensity variation with a DL that is significantly lower than that of the compositions outside the invention.

Composition P2 outside the invention, not comprising ferulic acid, becomes colourless under the same conditions.

Composition P3 outside the invention, not comprising ferulic acid and comprising ethylhexyl methoxycinnamate, itself becomes pale green under the same conditions. This is the sign of substantial yellowing of composition P3 outside the invention.

The spectrocolorimetric measurements confirm degradation of the colour intensity of compositions P2 and P3 outside the invention, which have DL values significantly higher than those of the composition according to the invention.

Thus, the composition according to the invention conserves its colour for a significantly longer time, without substantial yellowing.

In conclusion, the fragranced composition according to the invention, advantageously comprising a compound of formula (I) of natural origin, is more stable over time than a composition not in accordance with the invention, not comprising any compound of formula (I) or comprising a compound of synthetic origin.

1. Fragranced composition comprising:
a) at least one compound of formula (I), or a geometrical isomer thereof, an organic or mineral acid or base salt thereof, or a solvate thereof: 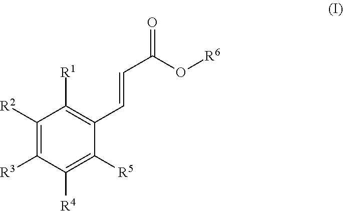
in which formula (I):
R¹, R⁴ and R⁵ represent, independently of each other, a hydrogen atom or a hydroxyl group;
R² represents a hydrogen atom, a hydroxyl group or a (C₁-C₆)alkoxy group;
R³ represents a hydroxyl group or a (C₁-C₆)alkoxy group; and
R⁶ represents an atom or a group chosen from: i) hydrogen, ii) (C₁-C₆)alkyl, optionally substituted with one or more groups chosen from hydroxyl, amino, carboxyl and/or amido, iii) cycloalkyl, optionally substituted with one or more groups chosen from (C₁-C₁₀)alkyl, (C₂-C₁₀)alkenyl, hydroxyl, amino, carboxyl and/or amido, and iv) glycosyl or glycosyloxy,
b) at least one dyestuff, and
c) at least one fragrancing substance.

2. Composition according to claim 1, said compound of formula (I) being chosen from ferulic acid and derivatives thereof.

3. Composition according to claim 1, said compound of formula (I) being chosen from natural compounds or compounds of natural origin.

4. Composition according to claim 1, comprising from 0.01% to 5.0% by weight of compound(s) of formula (I), relative to the total weight of said composition.

5. Composition according to claim 1, said dyestuff being chosen from water-soluble dyes, liposoluble dyes and natural pigments or pigments of natural origin.

6. Composition according to claim 1, said dyestuff being chosen from:
pyran or pyranium dyes or pigments;
polyene dyes or pigments 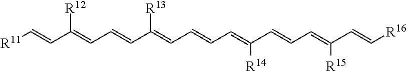
curcumin, the coloured active agents of which have the following formula: 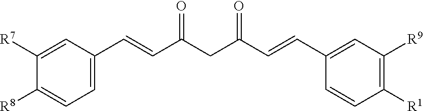
in which R⁷, R⁸, R⁹ and R¹⁰ represent, independently of each other, a hydrogen atom, a hydroxyl group or a (C₁-C₆)alkoxy it being understood that at least one of the groups R⁷ to R¹⁰ represents a hydroxyl group;
and also the geometrical isomers thereof, the organic or mineral base salts thereof, and the solvates thereof.

7. Composition according to claim 1, said dyestuff being chosen from pyran dyes or pigments, polyene dyes or pigments, extracts of red, blue or yellow gardenia, carotenes, paprika, curcumin, porphyrins and mixtures thereof.

8. Composition according to claim 1, comprising between 0.0001% and 20% by weight of dyestuffs(s) relative to the total weight of the composition containing the dyestuff(s) or the extract(s) containing same.

9. Composition according to claim 1, said fragrancing substance being chosen from essential oils, perfumes and aromas of synthetic or natural origin, and mixtures thereof.

10. Composition according to claim 1, comprising from 0.000100 to 10% by weight, relative to the total weight of said composition.

11. Composition according to claim 1, comprising one or more metal salts notably chosen from (poly)hydroxyalkyl(C₁-C₁₆)carboxylates of metals of oxidation state II, gluconates of metals of oxidation state II such as Fe, Cu, Zn or Mg gluconates, lactates of metals of oxidation state II such as Fe, Cu, Zn or Mg lactates.

12. Composition according to a claim 1, said composition being aqueous, alcoholic or aqueous-alcoholic.

13. Composition according to claim 1, which is in the form of an eau fraiche, an eau de toilette, an eau de parfum, an aftershave lotion, a care lotion, a silicone or hydrosilicone care oil, a fragranced two-phase lotion, a body milk or a shampoo.

14. Method for stabilizing a fragranced composition comprising at least one dyestuff, and at least one fragrancing substance, comprising at least a step of using a compound of formula (I), or a geometrical isomer thereof, an organic or mineral acid or base salt thereof, or a solvate thereof. 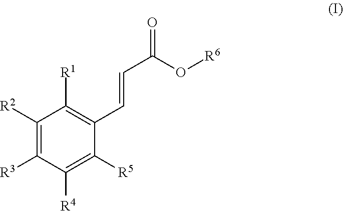
in which formula (I):
R¹, R⁴ and R⁵ represent, independently of each other, a hydrogen atom or a hydroxyl group;
R² represents a hydrogen atom, a hydroxyl group or a (C₁-C₆)alkoxy group;
R³ represents a hydroxyl group or a (C₁-C₆)alkoxy group; and
R⁶ represents an atom or a group chosen from: i) hydrogen, ii) (C₁-C₆)alkyl, optionally substituted with one or more groups chosen from hydroxyl, amino, carboxyl and/or amido, iii) cycloalkyl, optionally substituted with one or more groups chosen from (C₁-C₁₀)alkyl, (C₂-C₁₀)alkenyl, hydroxyl, amino, carboxyl and/or amido, and iv) glycosyl or glycosyloxy.

15. A method for treating keratin materials or clothing, comprising applying to the keratin materials and/or said clothing a fragranced composition comprising:
a) at least one compound of formula (I), or a geometrical isomer thereof, an organic or mineral acid or base salt thereof, or a solvate thereof: 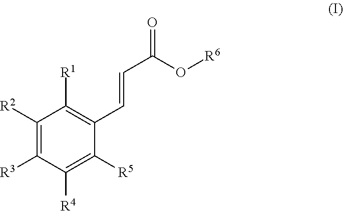
in which formula (I):
R¹, R⁴ and R⁵ represent, independently of each other, a hydrogen atom or a hydroxyl group;
R² represents a hydrogen atom, a hydroxyl group or a (C₁-C₆)alkoxy group;
R³ represents a hydroxyl group or a (C₁-C₆)alkoxy group; and
R⁶ represents an atom or a group chosen from: i) hydrogen, ii) (C₁-C₆)alkyl, optionally substituted with one or more groups chosen from hydroxyl, amino, carboxyl and/or amido, iii) cycloalkyl, optionally substituted with one or more groups chosen from (C₁-C₁₀)alkyl, (C₂-C₁₀)alkenyl, hydroxyl, amino, carboxyl and/or amido, and iv) glycosyl or glycosyloxy,
b) at least one dyestuff; and
c) at least one fragrancing substance.
